(19) United States
(12) Patent Application Publication
(10) Pub. No.: US 2003/0232368 A1
(43) Pub. Date: Dec. 18, 2003

(54) Method for detecting a molecular recognition by electrochemiluminescence

(75) Inventors: Martial Billon, Grenoble (FR); Gerard Bidan, Grenoble (FR); Agnes Dupont Filliard, Les Adreto (FR); Loic Blum, Caluire (FR)

Correspondence Address: OBLON, SPIVAK, MCCLELLAND, MAIER & NEUSTADT, P.C., 1940 DUKE STREET, ALEXANDRIA, VA, 22314, US

(73) Assignees: COMMISSARIAT A L'ENERGIE ATOMIQUE, PARIS (FR); UNIVERSITE JOSEPH FOURIER, Grenoble Cedex (FR); UNIVERSITE CLAUDE BERNARD LYON 1, Villeurbanne Cedex (FR)

(21) Appl. No.: 10/417,265

(22) Filed: Apr. 17, 2003

(30) Foreign Application Priority Data
May 3, 2002 (FR) 02 05562

(51) Int. Cl. C12Q 1/68; G01N 33/53; B05D 3/00

(52) U.S. Cl. 435/6; 435/7.1; 427/2.11

(57) ABSTRACT

The present invention concerns a method for detecting a molecular recognition by electrochemiluminescence. Said method comprises the steps consisting in a) depositing an electronically conductive film on a support and attaching on said film a sensor molecule in order to obtain a test support; b) subjecting the sample to be tested to a protocol of marking the target molecule with an electrochemiluminescent marker; c) bringing the marked target into contact with the test support; d) rinsing the test support; and e) subjecting the test support rinsed in step d) to a reading of the electrochemiluminescence signal triggered by a direct, or indirect, electronic transfer between the target and the support via the electronically conductive film.

### TECHNICAL FIELD

The present invention concerns a method for detecting a molecular recognition by electrochemiluminescence. In a general manner, it concerns the qualitative and quantitative detection of a specific molecular recognition between a first molecule attached on a support and a second molecule looked for in a sample.

According to the invention, specific molecular recognition may be defined as a specific interaction between two more or less complex molecules, leading to a bonding or assembly of the two molecules that is sufficiently stable to allow the molecules to be detected when they are linked together. In the present invention, this can involve, for example, a hybridisation of nucleic acids (DNA and/or RNA), an antigen/antibody type recognition reaction, a protein/protein type interaction, an enzyme/substrate type interaction, etc.

The method of the present invention finds an application, for example, in detection in the field of “Biochips” in the wide sense, in other words nucleic acid chips and protein chips or any systems including multi-array systems for the study of the sensor-target recognition of biological substrates. It involves, for example, the detection of hybridisation of nucleic acids on solid supports, in aqueous media or in the air, for example within the scope of a screening or a detection of hybridisation on a biochip.

The method of the present invention may be applied to any purposes of marking conductive surfaces by an electrochemiluminescent system.

### STATE OF THE PRIOR ART

Miniaturised biosensors or biochips are presently the subject of considerable economic interest. This interest is explained by the fact that they are able to carry out thousands of analyses in parallel and their extensive scope extending to the field of medical diagnosis, environmental and agri-business control and the pharmacology sector.

Said systems comprise sensor molecules, generally biological molecules such as fragments of DNA, or more generally fragments of oligonucleotides (or ODN), immobilised on very small surfaces. Said sensors, by their ability to specifically recognise given biological entities, called target molecules, such as complementary strands of DNA or ODN, impart to the biosensor a recognition selectivity.

The sensitivity of said biosensors depends in part on the technique used to reveal the phenomenon of sensor molecules/target molecules recognition. Numerous tools have been developed and more specifically some of these use fluorescent markers that offer a high detection sensitivity.

However, said techniques require a laser excitation of the fluorophores grafted onto the target biomolecules which may also be accompanied by a residual parasitic fluorescence from the illumination of the support and the biomolecules.

In the present description, the references between straight brackets [ ] refer to the appended list of references.

Electrochemiluminescence (ECL) or electrogenerated chemiluminescence is a phenomenon based on the emission of light via an electrochemical reaction. ECL has been employed as a means of detection [1] and [2]. Among the most commonly used electrochemiluminescent markers, one distinguishes organometallic type compounds such as the complex Ru(2,2′-bipyridine)₃²⁺ and organic compounds such as luminol and derivatives thereof.

Although designated in publications and reviews [1] by the same term ECL, said two families give rise to very different ECL mechanisms, in particular organometallic compounds are not used up by the reaction and the ECL emission is continuous, linked to the maintaining of a suitable electric potential and the presence of a co-substrate; whereas the luminol molecule, or derivatives thereof, is irreversibly consumed. We will talk in this case of electro-triggered chemiluminescence or ETCL.

Blackburn et al. [3] were the first to use detection by ECL in the case of the detection of products arising from the chain reaction of polymerase (PCR) by grafting beforehand the complex Ru(bpy)₃²⁺ onto proteins and nucleic acids. The detection limit was around subpicomolar with a linearity domain of more than six orders of magnitude. Moreover, said markers demonstrate very high stability and may be stored more than one year in the dark and at ambient temperature. Finally, it is possible to graft several Ru(bpy)₃²⁺ markers onto a same biomolecule without affecting its reactivity or its recognition properties. Xu et al. [4] have also applied said detection method by ECL to a DNA biosensor comprising simple strands of DNA immobilised on an electrode coated with a molecular assembly of aluminium alkane bisphosphonate. The hybridisation phenomenon between said immobilised fragments of DNA and complementary strands in solution has been highlighted via ECL of Ru(bpy)₃²⁺ complexes either inserted as intercalators or grafted beforehand onto the complementary stands.

Luminol has also been used as an ECL marker in an immunosensor used for the detection of 2,4-dichlorophenoxyacetic acid (2,4-D), a herbicide known for its potentially carcinogenic character [5]. 2,4-D in its activated ester form is anchored beforehand on a carbon electrode bearing aminohexane chains. Then by recognition of said herbicide by a luminol bearing antibody, it was possible to estimate the quantity of immobilised herbicide via the intensity generated by ECL of the luminophores in the presence of H₂O₂. Said immunodetection of the 2,4-D by ECL showed that it was possible to detect a limit concentration equivalent to 0.2 μgl⁻¹.

Unfortunately, said method cannot lead to a multi-array system. In fact, anchoring of the analyte 2,4-D is carried out in a non-specific manner on a self-assembled layer composed of aminohexane chains by simple chemical coupling between an amine function borne on one of the aminohexane chains and the acid function in its activated ester form borne on a 2,4-D molecule.

Electrochemiluminescence intercalators have been used to detect the hybridisation of DNA as described in document [6]. The method consists in immobilising the sample of target DNA on an electrode forming the base of an electrochemical cell then the DNA sensor and the ECL substance having specific bonding properties with a double strand are then added. Chemiluminescent intercalators such as acridine and lucigene have been used for ECL detection. In said document, the luminol which is not an intercalator of DNA was not used in an ETCL mechanism, but its chemiluminescence triggered by chemical methods, the luminol either being in solution, the sensor sequence being grafted by a peroxydase, or grafted on the sensor sequence.

However, the DNA/intercalator interaction is not always selective. Moreover, the intercalator interacts in a non-specific manner with single DNA sensor strands and is adsorbed on the film, which induces a parasite signal. As a result, said immobilisation method of the ECL sensor by intercalation does not allow a parallel and multi-array reading.

The techniques of the prior art are therefore not always precise, and, often, cannot be used in a multi-array system or in a MICAM chip type system, sold by the CisBio company (registered trademark).

### DESCRIPTION OF THE INVENTION

The precise aim of the present invention is to overcome the above-mentioned problems of the prior art by providing a method for detecting a molecular recognition between a sensor molecule attached on a support and a target molecule looked for in a sample to be tested, said method moreover allowing a precise and sensitive quantitative and qualitative detection of the target molecule when it is present in the sample.

The method of the present invention comprises the following steps:

a) depositing on a support an electronic or redox conduction electronically conductive film and attaching on said film a sensor molecule in order to obtain a test support,

b) marking, before or after step c), the target molecule with an electrochemiluminescent marker,

c) bringing the marked or non-marked target molecule into contact with the test support under physical and chemical conditions that allow the molecular recognition and the specific assembly between the sensor molecule and the target molecule,

d) rinsing the test support in such a way as to remove the excess of electrochemiluminescent marker while at the same time preserving the specific assembly between the sensor molecule and the target molecule from step c), and

e) subjecting the rinsed test support obtained in step d) to a reading of the electrochemiluminescence triggered by a direct, or indirect, electronic transfer between the target and the support via the electronically conductive film.

The detection of a chemiluminescence in step e) reveals an assembly by molecular recognition in the above-mentioned sense between the sensor molecule attached on the support and the target molecule looked for, and thus the presence of the target molecule in the tested sample.

In the method of the present invention, the detection of the recognition between the sensor molecule and the marked target molecule is based on electrogenerated chemiluminescence or electrochemiluminescence (ECL). It involves an optical detection which makes it possible to overcome the disadvantages of the methods of the prior art while at the same time conserving the sensitivity of optical detectors. In fact, the electric triggering of the chemiluminescence only concerns the chemiluminescent marker and not the test support or the biomolecules. Moreover, said novel detection method applied to biochips makes it possible to carry out quantitative measurements of the sensor molecules/target molecules recognition, unlike detection by fluorescence. Finally, the excitation by an electric impulse according to the present invention allows a spatial control and a rapid and simple use while at the same time not requiring a costly laser excitation device used in the prior art.

The present invention finds an application in the field of biochips, where the support on which is deposited the conductive film according to the present invention forms the biochip.

According to the invention, the support may be any of the supports used by those skilled in the art for the manufacture of biochips. By way of example and in nowise limitative the following may be used: a metal support such as Au, Pt, etc., a glass/ITO support, a metallised glass or quartz support, a plastic/ITO support, a metallised plastic support, a vitreous carbon support, or a support formed by the deposition of a conductive, screen-printed material on an insulating substrate or on one of the above-mentioned supports.

According to the present invention, the conductive film may be an intrinsic or redox electronically conductive film.

When it involves an electronically conductive film, it may be a film of conductive polymer, for example such as those used by those skilled in the art for the manufacture of biochips on which are grafted sensors. For example, the polymer may be a conductive polymer such as those described in “Techniques de l'Ingénieur”—A3140—under the denomination “intrinsic conductive polymers”: these are polymers formed from molecules bearing conjugated bonds and, if appropriate, doped with electron donor or acceptor dopants such as poly(acetylene), poly(sulphur nitride), polyphenylene, polypyrrole, poly(phenylene sulphide), polythiophene, polyaniline, etc.

According to the present invention, the conductive film may be deposited on the support using classical techniques known to those skilled in the art, for example electro-deposition or even electro-polymerisation.

According to the invention, the conductive film may be formed from simple pyrrole type monomers or pre-synthesised oligomers such as oligothiophenes, as well as from more complex molecular systems such as transition metal complexing units such as the phenantrolines, phenylpyridine, etc. bearing electropolymerisable units which leads, when the polymer is formed, to a conjugated system.

According to the present invention, when the electronically conductive film is a redox type, it may be a redox polymer comprising a poly(vinyl imidazole) type polymeric matrix containing redox centres such as a transition metal such as osmium, ruthenium, etc. complexed by bipyridines. An example of a poly(4-vinyl pyridine) containing a ruthenium complex having electrochemiluminescence properties that can be used in the present invention is described in [8].

According to the invention, the sensor molecule may be, for example, DNA, an oligonucleotide, an antibody, a protein or an enzyme, as well as any molecule or biomolecule allowing a specific recognition and an assembly as defined here above.

According to the invention, the attachment of the sensor molecule on the conductive film, when said film is a conductive polymer, may be carried out using the classical chemical techniques used to attach sensors to biochips. The bond between the support and the sensor may be via adsorption, electrostatic, chemical obtained by self-assembly, silanisation, or by any chemical, electrochemical, photochemical, etc. method known to those skilled in the art for depositing the sensor molecule for example functionalised by a group providing the self-assembly, anchoring, coupling property, or polymerisable chemically, electrochemically, photochemically or electrophotochemically through photosensitive reaction.

According to the invention, the bond between the film deposited on the support and the sensor may be obtained in one step for example by the MICAM process (registered trademark).

Documents FR-A-2 787 581, FR-A-2 787 582 and U.S. Pat. No. 5,810,989 describe for example techniques for film deposition and attaching sensor molecules that can be used in the present invention. It may involve, for example, electro-polymerisation on a support, for example on a silica substrate, of precursor molecules of the conductive polymer, such as pyrrole, with monomers, for example pyrrole, functionalised by a sensor molecule according to the present invention, for example oligonucleotides. In said techniques, one exploits the adhesion of the conductive polypyrrole film on the substrate in such a way as to achieve the attachment of the recognition sensor molecules.

According to the invention, the sensor may also be attached on the film deposited on the support for example by post-functionalisation of the film, for example of polypyrrole (Ppy), for example deposited by electrografting on the support, by an affinity recognition system, for example an avidin/biotin system or equivalent systems, or derivatives thereof.

This leads for example to the following conductor—sensor molecule film structures: Ppy-DNA; Ppy—biotin/avidin/biotin—DNA; Ppy—biotin/avidin protein; Ppy—biotin/avidin/biotin—antibody, that can be used according to the present invention. Said structures may for example be used on a gold (Au) or silica support, or any support that allows the grafting of the conductive polymer.

Said attachment may be carried out for example with addressing of the sensors. This involves for example photochemical addressing, mechanical addressing, for example by micropipetting using a disperser robot, and electrochemical addressing. Said techniques for attaching the sensor on a polymer film are known to those skilled in the art. The addressing allows multi-array analyses.

The anchoring of the sensor molecule may be achieved by simple coupling between two reactive chemical functions, for example activated ester and amine type, one of said functions being borne by the sensor molecule and the other anchored on the film. The sensor/film chemical coupling may be impeded by blocking the reactivity of the chemical function borne by the film. Said impediment may be lifted or “deblocked”, making the function once again reactive, thanks for example to an electrochemical or photochemical activation. At the end of this, the coupling of the sensor molecule on the film is once again possible.

According to the invention, the marker may be one of the electrochemiluminescent markers known to those skilled in the art. For example, it may be chosen from the group comprising luminol, isoluminol, an aminophthalhydrazine and derivatives thereof.

According to the invention, derivative of luminol or isoluminol are taken to mean derivative compounds with the following formulae:

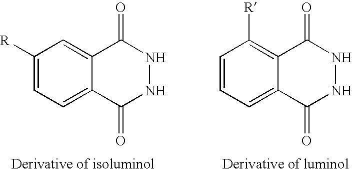

in which R or R′ are active groups that allow a functionalisation, in other words a chemical modification allowing the attachment of the marker on the target molecule in accordance with the present invention. By way of example, in nowise limitative, one may cite R or R′=H₂N —(H₂C)₃—NH; NH₂—(CH₂)₄—(C₂H₅)N; alkyl or alkoxy chains substituted or not and combinations and derivatives thereof. Said markers are available commercially, for example N-(4-aminobutyl)-N-ethylisoluminol under the trade name ABEI, manufactured by the SIGMA Company.

According to the invention, the bond between the target molecule and the luminol is formed through the R or R′ substituent. This is therefore chosen as a function of the reactivity of the target molecule.

The bond may be direct, for example DNA-luminol or protein-luminol, or indirect via a coupling through a biotin/avidin type affinity, for example DNA_{target}-biotin/avidin-luminol.

Marking protocols that may be used in the present invention are described for example in documents [9] and [10].

According to the invention, the marking of the target by the luminol may be before or after the step c) of bringing the target molecule into contact with the test support. In other words, the marking of the target molecule may be carried out either on the sample to be tested before it is brought into contact with the test support, in other words before the sensor molecule/target molecule assembly forms, or on the test support after the step c) of bringing the target molecule into contact with the test support; in other words, after the sensor molecule/target molecule assembly forms. Those skilled in the art will know how to adapt the method of the present invention for example depending on the type of sensor molecules/target molecules brought into play.

The step c) of bringing the target molecule into contact with the test support is obviously carried out under physical and chemical conditions that allow the molecular recognition and the specific assembly between the sensor molecule and the target molecule. Said conditions are for example those that allow the hybridisation of complementary nucleic acid strands in the case of nucleic acid sensor and target molecules, or those that allow a recognition and a protein/protein type interaction in the case of protein type sensor/target molecules. They are known to those skilled in the art.

The recognition between target molecule and sensor molecule during the step c) of bringing into contact leads to a specific sensor molecule/target molecule assembly: it involves a supramolecular or suprabiomolecular association resulting from a recognition or affinity between a sensor immobilised on the support and a target analyte in solution. Examples are cited above.

The step d) of rinsing consists in removing the excess of marker on the support, in other words the marker molecules not bonded to the target molecules. It may be carried out for example with physiological water or any other solution that preserves the sensor molecule/target molecule assembly on the support.

The step e) is an electrochemiluminescent reading. According to the invention, said luminescence is triggered by an electronic transfer between the marker of the target molecule and the electrically conductive support directly, or indirectly, via the electronically conductive film.

In said step, the support on which is deposited the conductive film serves as an electrode to circulate an electric current by applying a potential to the support in such a way as to trigger the electrochemiluminescence. The conductive film is obviously placed in a state of conductivity which allows or favours the above-mentioned transfer of electrons.

FIGS. 1 and 2 are schematic representations of a direct electronic transfer (FIG. 1) or indirect electronic transfer (FIG. 2) between the marked target molecule and the support via the electrically conductive film during an electrochemiluminescence reading according to the method of the present invention. In these figures, “su” represents the support, “f” the electronically conductive film, “li” the electronically conductive film/sensor molecule bond, “S” the sensor molecule, “C” the target molecule, “L” luminol, “e⁻” direct electronic transfer (FIG. 1) and “Mox/Mred” a redox pair called redox transmitter (M) (ox=oxidiser; réd=reducer) allowing an indirect electronic transfer through it.

According to the invention, the purpose of the redox transmitter between the support and the luminol is to transfer the electrons from the luminol towards the support. It may be in solution, or co-immobilised on the support with the assembly or linked to the assembly. By way of example in nowise limitative, it may be chosen from among: a metallocene group such as ferrocene or diferrocene, transition metal complexes such as a complex of cobalt, in solution or grafted onto the conductive film, for example of polypyrrole, or to the sensor, or intercalators of DNA having redox properties or comprising redox groups. Those skilled in the art will know how to adapt the method of the present invention as a function of the target and sensor molecules brought into play.

The final step of electrochemiluminescence reading may be carried out by means of a luminometer, which measures the intensity of luminescence emitted. The intensity of luminescence emitted is proportional to the concentration of target molecules assembled with the sensor molecules.

In the method of the present invention, the electronic transfer from the support towards the luminol is achieved through an electronically conductive film, which is not the case in the systems developed by Xu et al. [4] and Marquette et al. [5]. In fact, for said systems of the prior art, the anchoring of the electrochemiluminescent group on the support is achieved through respectively aluminium alkane biphosphonate and aminohexane, non-conductive layers.

The use of an electronically conductive film according to the present invention favours the support/luminol electronic transfer and thus the performance of said mode of detection by ECL.

Moreover, in the method of the present invention, the support has a double active function allowing (i) the electro-controlled immobilisation of the biological object within the electronically conductive film and (ii) the activation as a “trigger” for the ECL processes of the luminol, which allows it to be applied to a biochip type multi-array system (parallel analysis system) based on the technology developed over the last few years using a network of MICAM (trade name) type electrodes.

Every block comprising a MICAM type biochip that recognises in a specific manner a particular molecule, for example a DNA sequence, different molecules, for example different DNA sequences, present in a given sample may be detected simultaneously during the analysis. For the application of the present invention, it is sufficient to apply, either in a sequential manner, or in a parallel manner, to each of the blocks of such a biochip, the potential for triggering the ECL of the marker, for example luminol. Thus, for example for DNA, from the blocks that have a luminescence, it is possible to identify the DNA sequences present in the analyte. The support has a double active function allowing (i) the electro-controlled immobilisation of the biological sensor object, in this example DNA, and (ii) the activation as trigger for the ECL.

The present invention thus enables for the first time the implementation of an electrochemiluminescence method on an analysis system in parallel (multi-array) in which the immobilisation support is also that which allows the triggering of the chemiluminescence.

Moreover, the application of the present invention to a multi-array system allows a multi-array and/or in parallel reading that does not provide the successive positioning device described by the patent of Hashimoto et al. [6].

Moreover, the method of the present invention makes it possible to carry out direct quantitative analyses while at the same time eliminating the problems of background noise inherent in measurements by fluorescence.

Finally, in the present invention, the immobilisation of the luminol marker is carried out specifically on the target molecule, unlike the methods of the prior art which employ electrochemiluminescent intercalators in the case of DNA biosensors as described in document [6].

Other characteristics and advantages will become clearer to those skilled in the art on reading the examples that follow, given by way of illustration and in nowise limitative, and by referring to the appended figures.

### BRIEF DESCRIPTION OF THE DRAWINGS

FIGS. 1 and 2 are schematic representations of a direct electronic transfer (FIG. 1) and an indirect electronic transfer (FIG. 2) between the marked target molecule and the support via the electronically conductive film during an electrochemiluminescence reading according to the present invention.

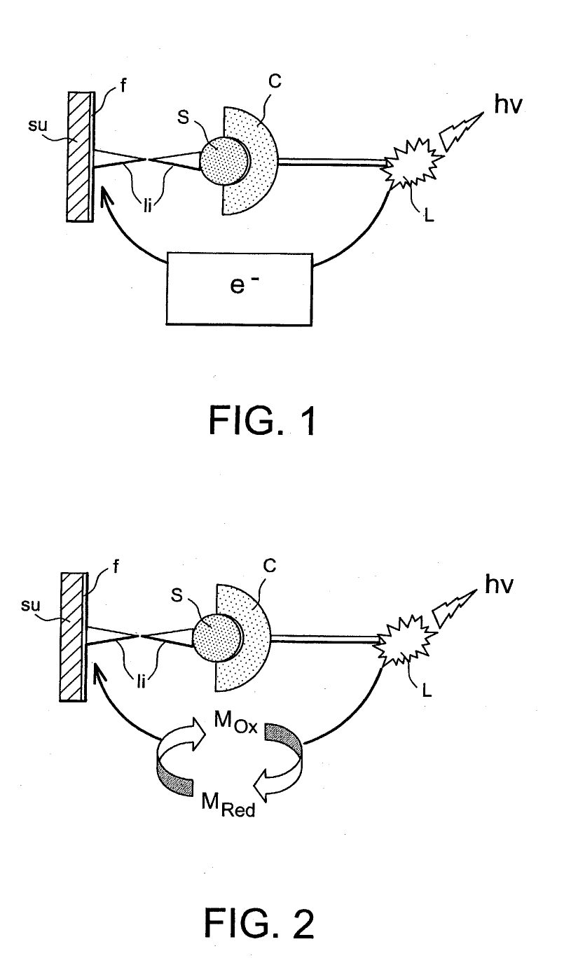

### EXAMPLES

### Example 1

Electrochemiluminescence of Luminol in Solution on an Electrode Coated with a Film of Polypyrrole

The inventors carried out beforehand a series of electrochemiluminescence (ECL) experiments in order to ensure that an electro-generated luminous emission could be activated from a support coated with an electronically conductive polymer such as polypyrrole, the electroluminescent group being in solution and not immobilised on the film of polypyrrole.

The ECL study was carried out from an electrode (“screen-printed”) composed of a working carbon electrode (S=0.2 cm2) and an AgCl/Ag reference electrode. The luminescence measurement was carried out using a luminometer which measures the luminescence intensity emitted from the “screen-printed” electrode placed in a measurement cell containing 2 ml of a solution of luminol of given concentration in a 30 mM Veronal-HCl, pH=9 buffered medium. The ECL measurement was carried out by firstly applying a potential of +0.450 V between the carbon electrode and the reference electrode then by injecting, into the previous mixture, 55 μl of a 2 mM hydrogen peroxide solution in a Veronal buffer medium. All of said operations were carried out under magnetic agitation and in a light protected environment.

The film of polypyrrole was electro-deposited beforehand on the support by electrochemical means at an applied potential of +0.80 V/ECS on the carbon electrode comprising the “screen-printed” electrode at a rate of 20 mC/cm² from a 50 mM solution of pyrrole monomer in 0.1 M H₂O/LiClO₄. Said electro-synthesis was carried out in a classical electrochemical cell. After it was formed, the film was then rinsed by soaking it in a 0.1 M H₂O/LiClO₄ monomer-free solution. At the end of said operation, the “screen-printed” electrode was placed in the measurement cell in order to carry out the electrogenerated luminescence measurement.

Under these conditions, it was possible to detect on the carbon electrode coated with a film of polypyrrole, a minimum quantity in luminol equal to 3 pmol (1.5 nM). For this quantity, the signal/noise ratio was less than 3, which indicated that the inventors had reached the detection limit.

When this ECL measurement was reproduced from a bare carbon electrode, in other words not coated with a film of polypyrrole, in contact with a Veronal buffer solution containing 1.5 nM luminol+55 μL in a 2 mM hydrogen peroxide solution, the collected luminous intensity was then 3 times higher than that measured previously.

It therefore appears that ⅔ of the luminescence is lost when the electrode is coated with a film of polypyrrole.

### Example 2

Electrochemiluminescence of Luminol in Solution on an Electrode Coated with a Film of Over-Oxidised Polypyrrole

In this example, the inventors carried out a luminescence measurement using a carbon electrode coated with a film of over-oxidised polypyrrole. The deposition of the polypyrrole was carried out in the same way as in Example 1.

The luminescence measurement from the ECL of the luminol in solution was carried out using the procedure described in Example 1.

However, in this example, before the ECL measurement and after the electro-synthesis of the film of polypyrrole, the carbon electrode coated with polypyrrole was subjected for 5 minutes to a potential of 1 V/ECS in 0.1 M H₂O/LiClO₄ medium. It has been shown in the literature that under these conditions the film of polypyrrole loses its electronic conductive properties (the polypyrrole is called over-oxidised).

The inventors observed that on said carbon electrode coated with a film of over-oxidised polypyrrole and from a Veronal buffer solution containing 3 nM of luminol+55 μl of a 2 mM solution of hydrogen peroxide, the application of a potential difference of 0.450 V did not lead to the emission of luminescence. Under these conditions, if the film of polypyrrole is over-oxidised beforehand, the phenomenon of ECL is then no longer detectable.

It follows from this experiment that the electronic conductive state of the film is important in the ECL process of luminol and that furthermore, the electrogenerated chemiluminescence takes place at the film/solution interface.

It should also be noted that no example in the literature mentions the observation of luminescence by ECL induced by electrochemiluminescent units, such as complexes of ruthenium trisbipyridine, immobilised in a film of polypyrrole.

### Example 3

ECL of Luminol Immobilised on a Film of Polypyrrole

A first ECL study of luminol immobilised on a film of polypyrrole was carried out using a commercially available derivative compound of luminol, streptavidin-isoluminol.

Streptavicin is a protein on which is grafted on average three molecules derived from isoluminol, ABEI (6-[N-(4-aminobutyl)-N-ethyl) amino-2,3-dihydro-1,4-phthalazine-1,4-dione). The streptavidin-isoluminol was anchored on a film of polypyrrole containing biotin entities (polypyrrole biotin) via the strong interaction existing between said molecules of biotin and streptavidin (affinity constant Ka=10¹⁵).

The film of polypyrrole biotin was electrosynthesized on the carbon electrode of the electrode “screen-printed” by successive scans with a potential of 50 mV/s betweeen −0.3 V and +0.8 V vs Ag+102 M/Ag from a 10 mM pyrrole biotin monomer (A) solution in 0.1 M CH₃CN/nBu₄PF₆ at a rate of 20 mC/cm².

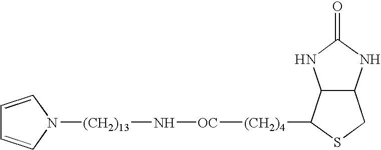

After rinsing the film in a PBS buffer solution pH=7, said film was brought into contact for 5 minutes with a PBS buffer solution pH=7 of streptavidin-isoluminol at 0.5 gl⁻¹. Then, the electrode was carefully rinsed with a 50 mM carbonate buffer solution pH=9.5.

The ECL measurement was carried out using said polypyrrole biotin/streptavidin-isoluminol molecular assembly.

As in the previous examples, the “screen-printed” electrode thus modified was placed in a measurement cell containing 2 ml of a 50 mM carbonate buffer solution pH=9.5. Then a potential difference of 0.450 V was applied between the carbon electrode and the AgCl/Ag electrode before injecting, into the carbonate buffer solution, 55 μl of a 2 mM hydrogen peroxide solution. The variation in the luminous intensity detected by photometer was then recorded.

After the addition of the hydrogen peroxide solution, a significant variation in the luminous intensity was recorded corresponding to 120 a.u. (arbitrary units), i.e. a quantity of immobilised streptavidin-isoluminol of around 20 pmol.cm².

Reproducing this experiment from a film of polypyrrole not containing biotin units, we were able to estimate the quantity of streptavidin-isoluminol simply adsorbed on such polymers. The non-biotinylated film was electrosynthesized from the pyrrole monomer designated (B):

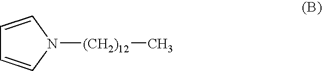

The measurement of the ECL intensity measured with said non-biotinylated film was 28 a.u., i.e. 5 pmol.cm⁻².

From these two measurements, one can thus estimate that the quantity of streptavidin-isoluminol specifically immobilised on the film of polypyrrole biotin was 15 pmol.cm⁻².

This value was in agreement with the value obtained by quartz microbalance measurements.

### Example 4

Detection by Electrochemiluminescence of the Complementary ODN/ODN Recognition on a Film of Polypyrrole Biotin

In this example, the inventors show that the recognition between an immobilised ODN sensor and a complementary ODN target may be detected via ECL by electrochemiluminescence.

The ECL sensor, here the streptavidin-isoluminol, is coupled onto the target analyte, namely the complementary ODN. As for the ODN sensor, it is immobilised on a film of polypyrrole biotin through the intermediary of previously anchored avidin molecules. Thus the molecular assembly used in this example corresponds to the multilayer system: polypyrrole biotin/avidin/ODN sensor-ODN target/streptavidin-isoluminol.

First of all, a film of polypyrrole biotin was electrosynthesized on the support in accordance with the operating conditions described in Example 3. After soaking in a PBS buffer solution, said film was then left for 5 minutes in a 0.125 g.l⁻¹ solution of avidin in a PBS medium, which led to the immobilisation of the avidin molecules on the film of polypyrrole biotin via the strong biotin/avidin interaction. At the end of this, the electrode was rinsed with a PBS buffer solution and then brought into contact with a 0.6 μM biotinylated ODN sensor solution.

Due to the fact that each ODN sensor bears at its end a biotin function, their anchoring on the polypyrrole biotin/avidin assembly comes about easily by simple bringing into contact allowing the interaction between the avidin sites that have remained free and the biotin molecules grafted onto the OND sensors.

The recognition of the ODN targets by the ODN sensors immobilised on the polypyrrole biotin/avidin/ODN sensor architecture. As previously, this was achieved simply by bringing into contact said molecular architecture with a 0.6 μM solution of complementary ODN target (ODN also biotinylated) in PBS medium. In order to ensure by ECL that the hybridisation between the ODN sensors and the ODN targets was indeed effective, a molecule of streptavidin-isoluminol was then grafted onto the ODN target matched to the ODN sensor. Here again, said grafting is simply achieved by simple biotin/avidin interaction, knowing that the ODN target bears at its end a biotin molecule.

Before carrying out the ECL measurement itself, the polypyrrole biotin/avidin/ODN sensor-ODN target/streptavidin-isoluminol assembly anchored on the carbon electrode of a “screen-printed” electrode was carefully rinsed with a 50 mM carbonate buffer solution pH=9.5. Then, as previously, the ECL measurement was carried out by placing the “screen-printed” electrode in the measurement cell containing 2 ml of the carbonate buffer solution pH=9.5. After injecting, into this medium, 55 μl of a 2 mM hydrogen peroxide solution, a potential difference of 0.450 V was applied between the carbon electrode and the AgCl₍ₛ₎/Ag reference electrode. The measurement of the luminous intensity emitted by ECL was on average 65 a.u., which corresponds to a surface immobilisation of streptavidin-isoluminol equal to 10 pmol cm⁻². From this result, it follows that the detection and the quantification of a biological event such as hybridisation between an ODN target and a complementary ODN target taking place at the interface of an electrically conductive support may be achieved by ECL.

In order to ensure that the electrochemiluminescence observed was indeed due to the hybridisation between the ODN sensor/ODN target oligonucleotides, the inventors carried out a similar experiment where, in this case, the polypyrrole biotin/avidin/ODN target assembly was brought into contact with a solution containing not complementary ODN targets but simply non-complementary ODN. In this case, no specific recognition by hybridisation was produced between the immobilised ODN sensors and the non-complementary ODN in solution.

As a result, the streptavidin-isoluminol could then immobilise itself on the polypyrrole biotin/avidin/ODN target system since there were no biotin anchoring points. For this reason, as expected, no luminescence was detected in this case after injecting 55 μL of the 2 mM hydrogen peroxide solution and the application of the potential, thus confirming that the result observed previously was indeed the result of hybridisation between the ODN sensor and the complementary ODN target.

### Example 5

Reproducibility of the Detection by ECL of Complementary ODN/ODN Recognition on a Film of Polypyrrole Biotin

As shown in document [7], it is possible, with the aid of a powerful detergent, sodium dodecylsulphate (SDS), to destroy the biotin/avidin interactions, which induces the regeneration of the polypyrrole-biotin polymer matrix.

The inventors therefore studied the possibility of reusing for a second time the polypyrrole biotin/avidin/ODN sensor-complementary ODN target/streptavidin-isoluminol assembly, for another ECL measurement.

For this reason, after the first ECL measurement made on the polypyrrole biotin/avidin/ODN sensor-complementary ODN target/streptavidin-isoluminol assembly, said assembly was treated with a 50 mM aqueous solution of SDS at 60° C. in order to destroy said molecular architecture and to be able to re-find only the polypyrrole biotin film anchored on the carbon electrode.

After a careful rinsing with de-ionised water, a new assembly identical to the first (polypyrrole biotin/avidin/ODN sensor-complementary ODN target/streptavidin-isoluminol) was formed on said film of regenerated polypyrrole biotin.

Once said new assembly had been formed, the luminophor was excited as previously under a potential of 0.450 V by plunging the electrode into a medium composed of 2 ml of 50 mM carbonate buffer solution pH=9.50, 55 μl of 2 nM hydrogen peroxide solution. The evolution of the luminous intensity recorded during this experiment gave a variation of 55 a.u. Said value was slightly inferior to that obtained during the first measurement and corresponds to a concentration in streptavidin-isoluminol of 8 pmol cm².

This study shows the possibility of detecting once again the hybridisation of the DNA after regeneration of the polypyrrole biotin matrix.

### Bibliographic References

[1] Fahnrich K. A., Pravda M. and Guibault G. G., “Recent applications of electrogenerated chemiluescence in chemical analysis” Talanta 2001, 54(4), 531-559.

[2] U.S. patent WO 9639534 “Electrochemiluminescence enzyme biosensors” Martin M. T.

[3] Blackburn G. F., Shah H. P., Kenten J. H., Leland J., Kamin R. A., Link J., Peterman J., Powell M. J., Shah A., Talley D. B., Tyagi S. K., Wilkins E., Wu T. G. and Massey R. J., Clin. Chem 1991, 37, 1534.

[4] U.S. patent WO 9606946 “Biosensor for and method of electrogenerated chemiluminescent detection of nucleic acid adsorbed to a solid surface” Bard A. J. and Xu X-H.

[5] Marquette C. A. and Blum L. J., “Electrochemiluminescence of luminol for 2,4-D optical immunosensing in a flow injection analysis system”, Sensors Actuat. B 1998, 51, 100-106.

[6] U.S. Pat. No. 5,776,672 Jul. 7, 1998, “Gene detection method”, K. Hashimoto, K. Ito, Y. Ishimori and M. Gotoh.

[7] A. Dupont-Filliard, A. Roget, T. Livache and M. Billon, “Reversible oligonucleotide immobilization based on biotinylated polypyrrole film”, Anal. Chem. Acta 2001, 449, 45-50.

[8] R. J. Foster and C. F. Hogan, Anal. Chem. 2000 (72), 5576.

[9] Analytical Chemistry Vol. 48, n° 13, November 1976, pp. 1933.

[10] Analytical Biochemistry, 111, 87-96 (1981), pp 87.

1. Method for detecting a molecular recognition between a sensor molecule attached on a support and a target molecule looked for in a sample to be tested, said method comprising the following steps:
a) depositing on a support an electronic or redox conduction conductive film and attaching on said film a sensor molecule in order to obtain a test support,
b) marking, before or after step c), the target molecule with an electrochemiluminescent marker,
c) bringing the target molecule into contact with the test support under physical and chemical conditions that allow the molecular recognition and the specific assembly between the sensor molecule and the target molecule.
d) rinsing the test support in such a way as to remove the excess of electrochemiluminescent marker while at the same time preserving the specific assembly between the sensor molecule and the target molecule from step c), and
e) subjecting the rinsed test support obtained in step d) to a reading of the electrochemiluminescence signal triggered by a direct, or indirect, electronic transfer between the target and the support via the electronically conductive film.

2. Method according to claim 1, in which step a) is an electro-polymerisation, on the support, of precursor monomers of the conductive polymer with precursor monomers functionalised by the sensor molecule.

3. Method according to claim 1, in which the electronically conductive film is a film of polypyrrole.

4. Method according to claim 1, in which the sensor molecule is attached on the conductive film by an avidin/biotin system.

5. Method according to claim 1, in which the sensor molecule is DNA, an antibody, a protein or an enzyme.

6. Method according to claim 1, in which the electrochemiluminescent marker is chosen from the group comprising luminol, isoluminol, an aminophthalhydrazine and derivatives thereof.

7. Method according to claim 1, in which the electrochemiluminescent marker is chosen from the group comprising derivatives of luminol or isoluminol with the following formulae: 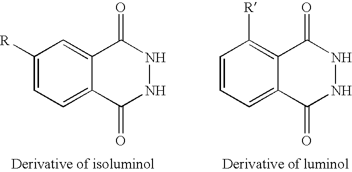
in which R or R′ are active groups that allow the marking of the target molecule, chosen from H₂N—(H₂C)₃—NH; NH₂—(CH₂)₄—(C₂H₅)N or derivatives thereof.

8. Method according to claim 1, in which, in step e), since the electronic transfer between the target and the support via the electronically conductive film is indirect, it is achieved by means of a redox transmitter chosen from among a metallocene group such as ferrocene or diferrocene, transition metal complexes such as a complex of cobalt, in solution or grafted onto the conductive film or onto the sensor molecule, or intercalators of the sensor molecule having redox properties or comprising redox groups.

9. Use of a method according to any of claims 1 to 8, for a quantitative or qualitative analysis of a target molecule present in a sample.

10. Use of a method according to any of claims 1 to 9, in a multi-array and/or parallel analysis system.
